(19) United States
(12) Patent Application Publication
(10) Pub. No.: US 2026/0257030 A1
(43) Pub. Date: Sep. 3, 2026

(54) TARGETED ALTERNATING PEEP VENTILATION (TAPV) STRATEGY USING PEAK RANGE

(71) Applicants: Filip Depta, Kosice (SK); Michael Anthony Anthony, Hillsborough, NC (US); Neil Russell EULIANO, II, Newberry, FL (US)

(72) Inventors: Filip Depta, Kosice (SK); Michael Anthony Anthony, Hillsborough, NC (US); Neil Russell EULIANO, II, Newberry, FL (US)

(21) Appl. No.: 18/866,259

(22) PCT Filed: May 17, 2023
(86) PCT No.: PCT/US2023/067103

§ 371 Date: Nov. 15, 2024

(51) Int. Cl. A61M 16/00

(52) U.S. Cl. CPC A61M 16/026

(57) ABSTRACT

A system and method is claimed for mechanically ventilating a patient including a breath delivery system. The system includes at least one airway sensor and a computing means, wherein the breath delivery system provides a flow of gas to the patient, the airway sensor measures pressure and flow of the gas leaving the patient. The computing means calculates an expiratory time constant. The breath delivery system provides PEEP to the patient, wherein the computing means determines a PEEP level or range of values based on an evaluation of the expiratory time constant or functions of the expiratory time constant at several PEEP levels.

### CROSS-REFERENCE TO RELATED APPLICATIONS

This application claims priority from and is related to PCT Application No. PCT/US2023/67103, entitled “Targeted Alternating PEEP Ventilation (TAPV) Strategy Using Measured Dynamic Expiratory Time Constant” with attorney docket number 9310-10001-WO, filed May 17, 2023, which claims priority from and is related to U.S. Provisional Patent Application No. 63/342,702, entitled “Targeted Alternating PEEP Ventilation (TAPV) Strategy Using Measured Dynamic Expiratory Time Constant” with attorney docket number 9310-V0001, filed May 17, 2022, which is hereby incorporated into the present application by reference in its entirety.

### BACKGROUND

The present invention relates to unique methods of monitoring, controlling and assessing ventilator flows to a patient, in particular, a method and system to ventilate nonhomogenous lungs, which the inventors call Targeted Alternating PEEP Ventilation (TAPV). Typically, a patient requiring respiratory support is connected to a mechanical ventilator that either includes or is monitored externally with airway flow and pressure sensor(s) attached to the airway tubing between the ventilator and the patient. These sensors measure the flow, pressure, volume, and other parameters that can be calculated from these sensors. These raw signals may be preprocessed in a computing means to calculate additional parameters, clean the signal, remove sensor biases and offsets, etc. The ventilator breath delivery system provides a source of pressurized gas to the patient to assist the patient in breathing. The ventilator usually delivers a baseline pressure of gas to hold the patient's airways open, which is called Positive End Expiratory Pressure (PEEP). The strategy for setting optimal PEEP is a controversial but important topic. The ventilator often measures parameters including tidal volume (Vt, the volume of air inhaled or exhaled in a given breath), flow rate (the rate at which flow is delivered to the patient), inspiratory time (the time required to deliver the breath), expiratory time (the time between inspirations that allows the patient to exhale), inspiratory pressure (the pressure at which air is delivered to the patient), peak inspiratory pressure (the highest pressure of air delivered to the patient), PEEP level (the measured or setting for PEEP), breathing frequency (number of breaths per minute), and other parameters.

Mechanical ventilation is essentially a dynamic process. However, previous research studies have focused mostly on static parameters when evaluating lung recruitment (i.e., lung response to the delivered Vt). Most of the ventilatory parameters set by clinicians (e.g., mode of ventilation, inspiratory flow, set pressures, and inspiratory to expiratory [I:E] ratio) affect the lung response to the delivered Vt, including pulmonary compliance which is commonly used for PEEP titration. On the other hand, the expiratory lung mechanics are less prone to variation due to set ventilatory parameters but have been scarcely used to assess the lung function to date.

Critical care of the future will be personalized for each patient. Mechanical ventilation (MV) is an integral part of this process. Continuous monitoring and assessment of the patients' lung condition is required, along with individualized adjustments to optimize care. That is the reason why measured and frequently evaluated respiratory mechanics parameters must be considered when initiating and managing MV, rather than using static and conventional recommendations applied uniformly.

To accomplish this, we decided to use exhalation dynamics and measured time constant (RC_{EXP}) to establish a ventilation strategy that uses alternating levels of PEEP and the predicted optimal ventilation frequency that are based on the measured lung mechanics of each individual patient. Passive exhalation dynamics are less prone to variation due to set ventilatory parameters but have been scarcely used to assess the lung function to date.

### SUMMARY OF THE INVENTION

In general, a novel method and apparatus to create a new personalized ventilation strategy for nonhomogenous lungs named Targeted Alternating PEEP Ventilation (TAPV), performs the following:
1. Determine the response to PEEP and detect the most optimal PEEP level using RC_{EXP}
2. Determine recruitability and recruitable zone by determining PEEP range using RC_{EXP}
3. Determine optimal ventilation frequency using RC_{EXP}
4. Determine complete exhalation time using RC_{EXP}
5. Calculate and adjust the I:E ratio to minimize intrinsic PEEP (PEEPi) using RC_{EXP}
6. Verify recruitability by performing series of expiratory holds using RC_{EXP}
7. Calculate exhalation angle (EA) using RC_{EXP}
8. Calculate peak expiratory flow to RC_{EXP} ratio at first, second and third RC_{EXP}
9. Determine most appropriate PEEP shifting based on the reconstructed model of real patients' physiology
10. Integrate all aspects mentioned above into the complex TAPV strategy

The TAPV strategy can optimize ventilation to obtain the safe, effective ventilation by providing appropriate pressure, flow, and volume and ideally reaching the lowest acceptable pressure, volume, PEEP, and respiratory rate to maximize safety while providing adequate ventilation and oxygenation to the patient.

### BRIEF DESCRIPTION OF THE SEVERAL VIEWS OF THE DRAWINGS

The accompanying figures wherein reference numerals refer to identical or functionally similar elements throughout the separate views, and which together with the detailed description below are incorporated in and form part of the specification, serve to further illustrate various embodiments and to explain various principles and advantages all in accordance with the present invention, in which:

FIG. 1 shows a scheme of Targeted Alternative PEEP Levels (TAPV) strategy. For illustration, three levels of PEEP are used and supported and/or unsupported breath is delivered at each PEEP level.

FIG. 2A is an illustration of the first measured expiratory time constant (RC_{EXP}) from expiratory flow waveform during expiration with regard to the percentage of exhaled volume, i.e. first RC_{EXP} equals to 63% of exhaled Vt.

FIG. 2B is an illustration of the first measured expiratory time constant (RC_{EXP}) from volume vs. time waveform corresponding to 63% of exhaled Vt.

FIG. 2C is an illustration of the first, second and third RC_{EXP} measured from expiratory flow waveform corresponding to 63, 86 and 95% of exhaled Vt, respectively.

FIG. 3A and FIG. 3B shows an example of PEEP titration using constant inspiratory pressure of 14 cmH₂O during pressure-controlled ventilation (PCV) in two different passive severe COVID-19 ARDS patients. The set of shaded boxes points out similar lung mechanics at different PEEP levels (i.e., PEEP range). Vte—expiratory tidal volume C_{RS}—respiratory system compliance, Vd/Vt—dead space fraction, VA—alveolar ventilation, PEEP—positive end-expiratory pressure.

FIG. 4A through FIG. 4F is a series of prone versus supine plots showing the optimal PEEP range (indicated by shaded boxes) according to the maximum expiratory time constant (RC_{EXP})±5%. NR—non-recruitable pattern, R—recruitable pattern, PEEP—positive end-expiratory pressure.

FIG. 5 is an example of PEEP titration method to determine PEEP levels using highest value of RC_{EXP}, according to one aspect of the present invention.

FIG. 6 is a schematic representation of the flow vs time curve during PCV breath. The expiratory flow curve (darker solid line) shows incomplete exhalation (PEEPi), and the dashed line depicts the predicted respiratory rate based on three-time constants (f_{P}). The predicted time cycle (T_{CYP}) and predicted inspiration-to-expiration ratio (IE_{P}) are also shown. RC_{EXP1}, RC_{EXP2}, RC_{EXP3} represent measured first, second and third time constant. PCV—pressure-controlled ventilation, V′—flow (liters per minute), RR—respiratory rate on the ventilator, IE—inspiratory-to-expiratory time ratio, T_{CY}—time of the breath cycle, Ti—inspiratory time, Te—expiratory time, PEEPi—intrinsic PEEP, I—inspiration, E—exhalation, Ep—predicted exhalation.

FIG. 7 shows an example of a series of inspiratory holds. At least two end-inspiratory holds of different durations are applied, and RC_{EXP} is evaluated to evaluate the recruitability of the lungs.

FIG. 8 shows the exhalation angle-connection of the first expiratory time constant with peak expiratory flow rate (PEFR). “Sharp” angle means that the PEFR is high and/or RC_{EXP} is short that would suggest a presence of “stiff lungs”. The opposite is true for compliant lungs and or obstructive conditions.

FIG. 9 shows examples of different EA (exhalation angle) scenarios: A—airway obstruction (i.e., COPD) with a long time constant and a low PEFR, B—normal patient, C—inappropriately high PEEP featuring short time constant due to overdistention and high PEFR.

FIG. 10 shows PEFR₁ at RC_{EXP1} (Vₘₐₓ₁/RC_{EXP1}), PEFR₂ at RC_{EXP2} (Vₘₐₓ₂/RC_{EXP2}), and PEFR₃ at RC_{EXP3} (Vₘₐₓ₃/RC_{EXP3}) corresponding to the flow at first, second or third RC_{EXP}.

FIG. 11A through FIG. 11D are examples of different suggested PEEP shifting patterns.

FIG. 12 is a proposed flow diagram of the targeted alternating PEEP ventilation (TAPV), according to one aspect of the present invention.

FIG. 13 is a diagram of the system illustrating the sensors, are connected either wirelessly or wired to a module that transmits the data to a computer or ventilator.

### DETAILED DESCRIPTION

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting but merely as a basis for the claims and as a representative basis for teaching one skilled in the art to variously employ the present subject matter in virtually any appropriately detailed structure and function. Further, the terms and phrases used herein are not intended to be limiting but rather, to provide an understandable description of the concepts.

The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The method and apparatus, according to the invention create a new personalized ventilation strategy for nonhomogenous lungs named Targeted Alternating PEEP Ventilation (TAPV).

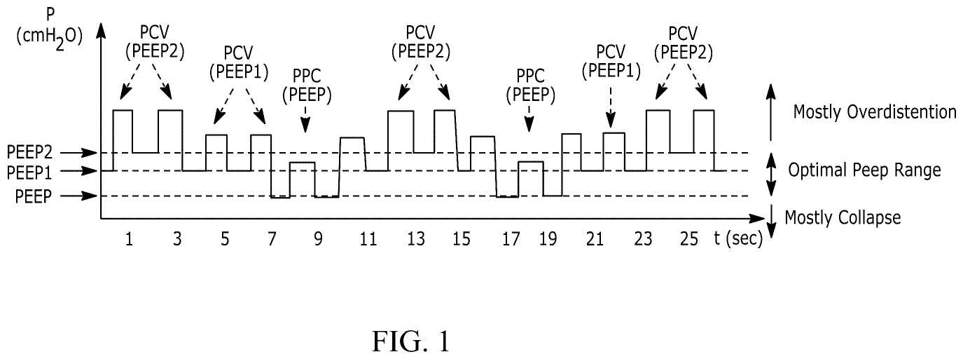

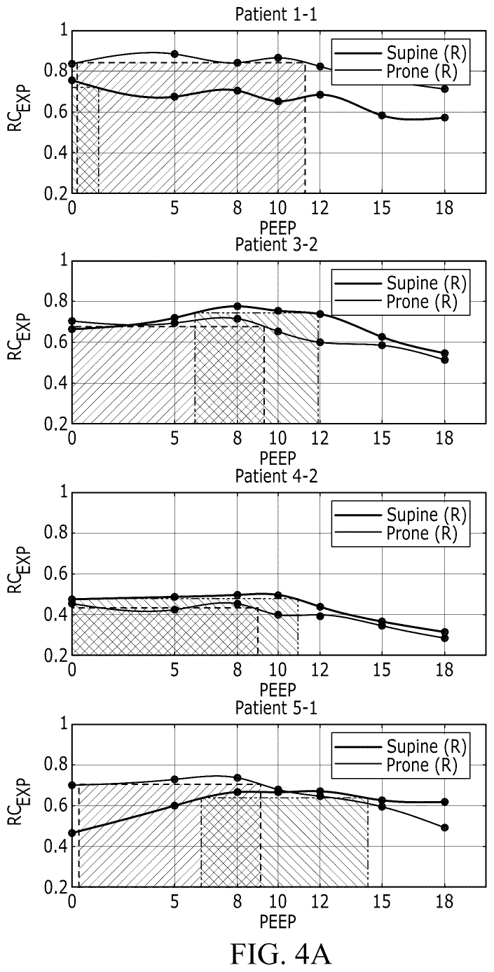

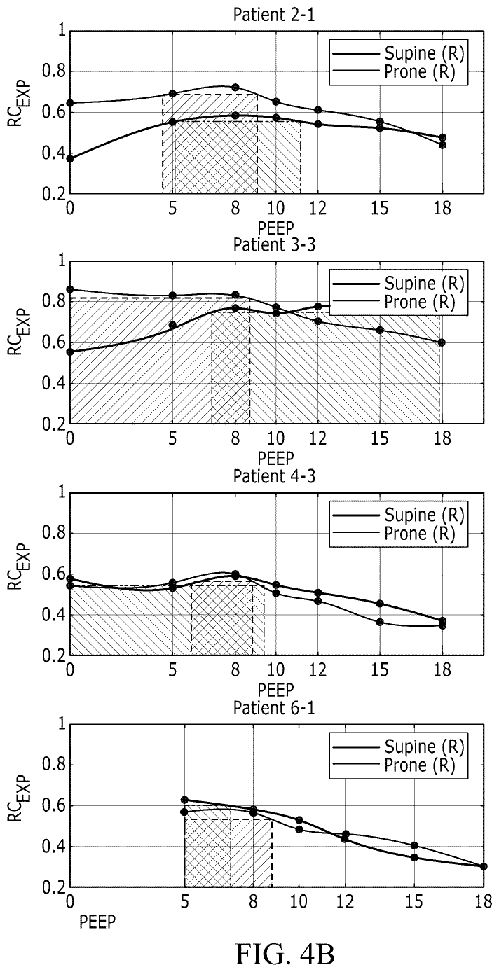

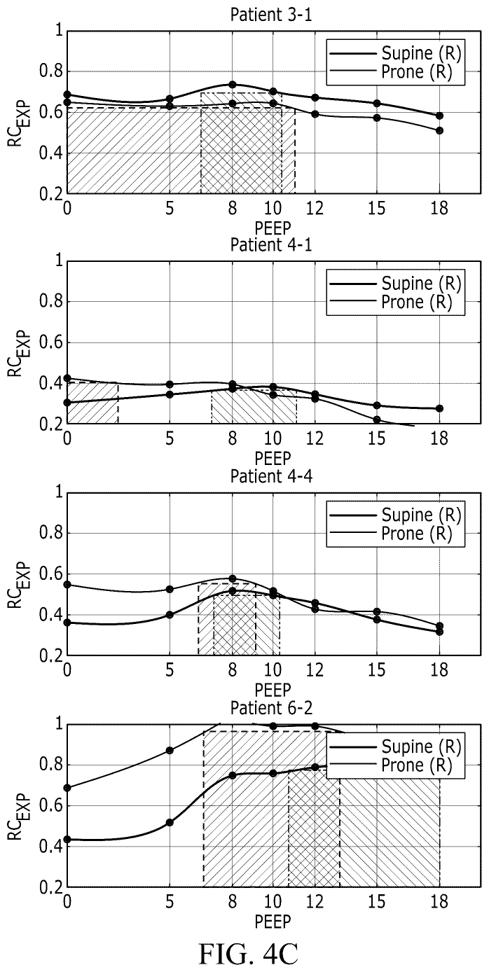

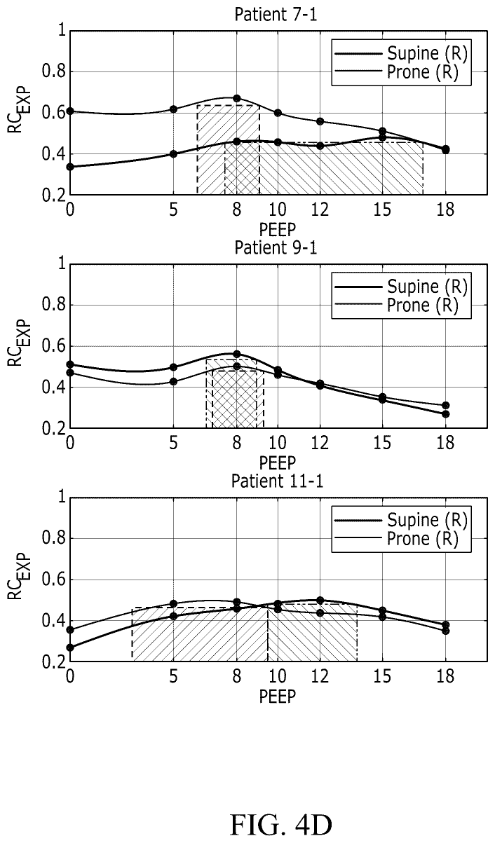

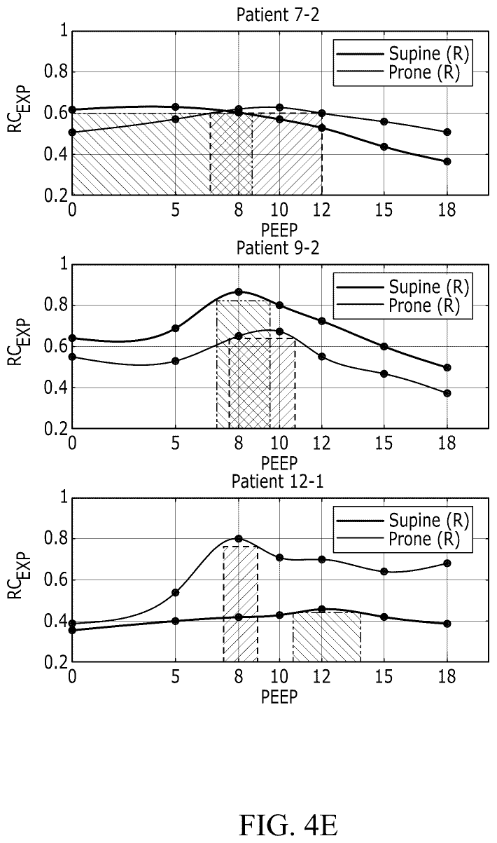

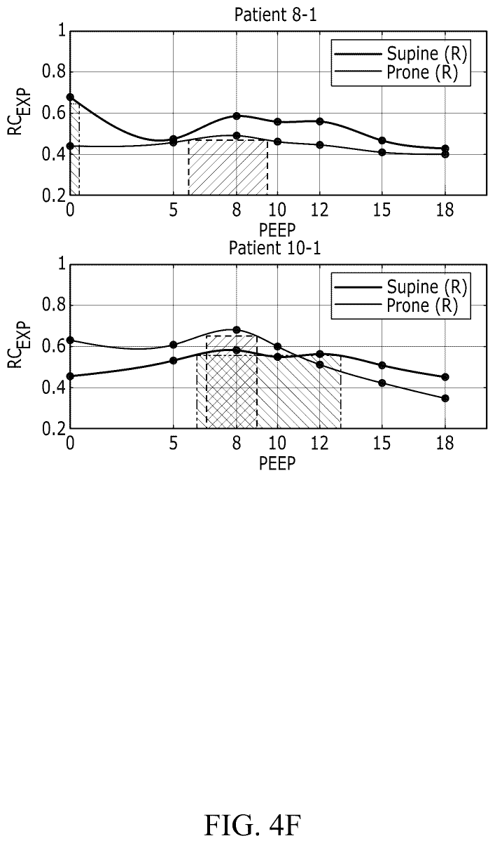

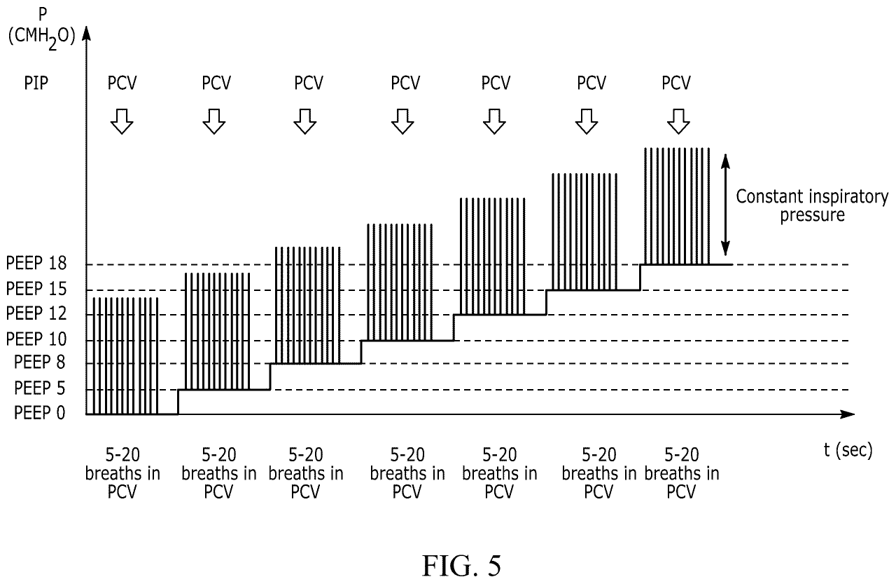

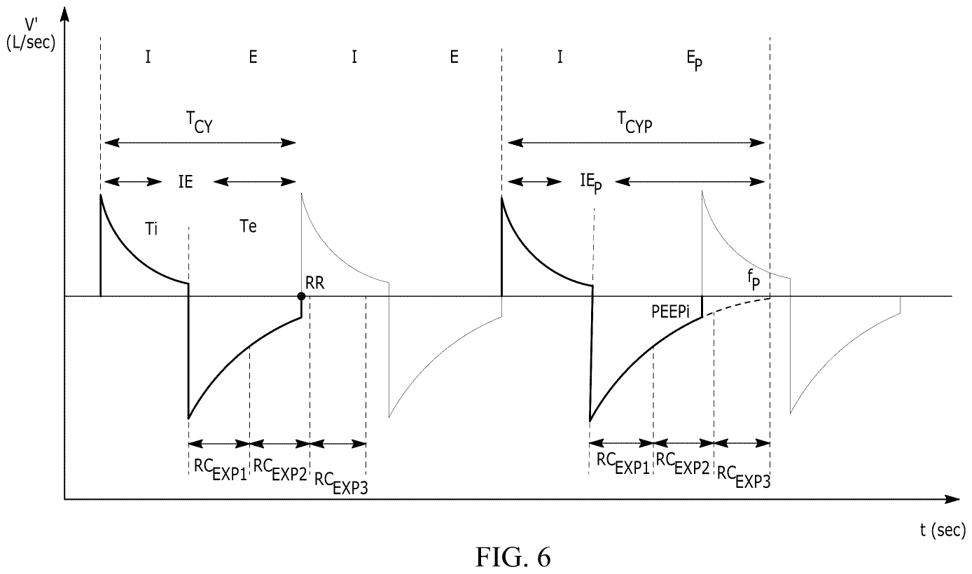

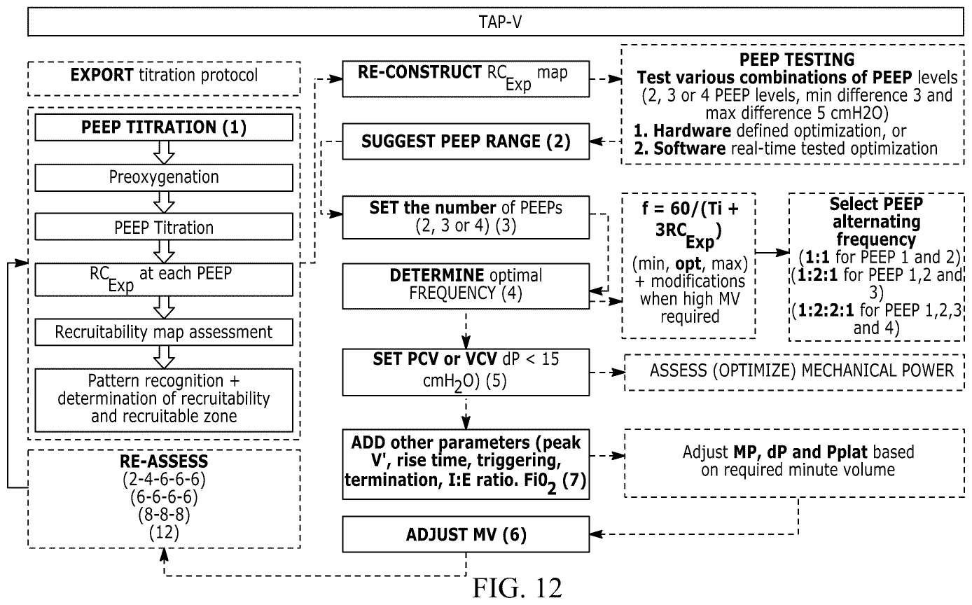

In general, concepts for such ventilation support can be summarized as follows (FIG. 12):
1. Using measured expiratory time constant (RC_{EXP}) to determine the optimal PEEP levels (similar to the highest static compliance, lowest dead space fraction or the best oxygenation) (FIG. 4).
2. Utilization of quick ‘diagnostic’ PEEP titration using constant inspiratory pressure, to identify PEEP range—the range of pressures where mechanical lung properties are similar, thereby assessing lung recruitability (FIG. 5).
3. Based on PEEP titration results for each individual patient and the exhalation mechanics obtained, we drafted the concepts of targeted alternating PEEP levels (FIG. 1).
4. Using the expiratory time constant, we aim to predict the optimal ventilation frequency where intrinsic PEEP (PEEPi) is minimized (FIG. 6).

### INCORPORATED REFERENCES

The following references are incorporated by reference in their entirety:
Filip Depta et al., Should we continue searching for the single best PEEP?. ICMx 10, 9 (2022).
Filip Depta et al., Time constant to determine PEEP levels in mechanically ventilated COVID-19 ARDS: a feasibility study. BMC Anesthesiol 22, 387 (2022)
Filip Depta et al., Determining respiratory rate using measured expiratory time constant: A prospective observational study. J Crit Care. 2023 February; 73:154174.

### Non-Limiting Definitions

Generally, the terms “a” or “an”, as used herein, are defined as one or more than one. The term plurality, as used herein, is defined as two or more than two. The term “adapted to” describes the hardware, software, or a combination of hardware and software that is capable of, able to accommodate, to make, or that is suitable to carry out a given function. The term “another”, as used herein, is defined as at least a second or more.

The phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N, that is to say, any combination of one or more of the elements A, B, . . . or N including any one element alone or in combination with one or more of the other elements which may also include, in combination, additional elements not listed.

As used herein, the term “about” or “approximately” applies to all numeric values, whether or not explicitly indicated. These terms generally refer to a range of numbers that one of skill in the art would consider equivalent to the recited values (i.e., having the same function or result). In many instances, these terms may include numbers that are rounded to the nearest significant figure. As used herein, the terms “substantial” and “substantially” mean, when comparing various parts to one another, that the parts being compared are equal to or are so close enough in a dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

The term “computer” or “computing means” is used to describe any computer processor executing computing instructions, including a mobile phone, tablet, laptop, desktop computer, as a web service or ventilator, when connected to the sensors to carry out the method of the present invention.

The term “configured to” describes hardware, software or a combination of hardware and software that is adapted to, set up, arranged, built, composed, constructed, designed, or that has any combination of these characteristics to carry out a given function.

The term “connected” or “coupled” means an element is connected to another element, it can be directly connected or coupled to the other element or intervening elements may be present.

In contrast, when an element is referred to as being “directly connected” or “directly coupled” to another element, there are no intervening elements present.

The terms “including” and “having,” as used herein, are defined as comprising (i.e., open language).

The term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

It should be understood that the steps of the methods set forth herein are not necessarily required to be performed in the order described, and the order of the steps of such methods should be understood to be merely exemplary. Likewise, additional steps may be included in such methods, and certain steps may be omitted or combined in methods consistent with various embodiments of the present device.

Although the elements in the following method claims, if any, are recited in a particular sequence with corresponding labeling unless the claim recitations otherwise imply a particular sequence for implementing some or all of those elements, those elements are not necessarily intended to be limited to being implemented in that particular sequence.

### Measured Expiratory Time Constant Considerations

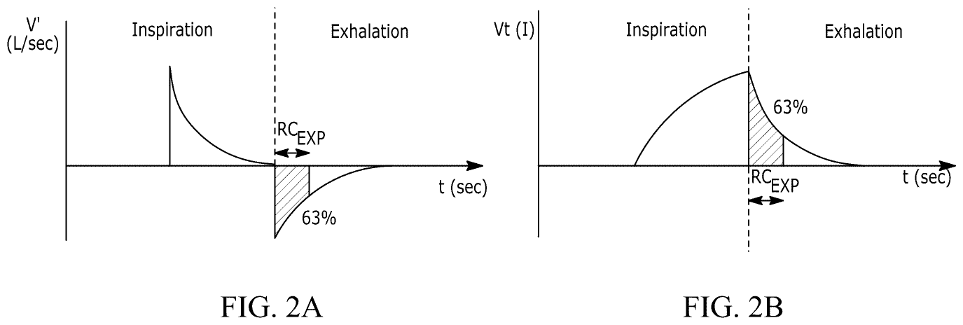

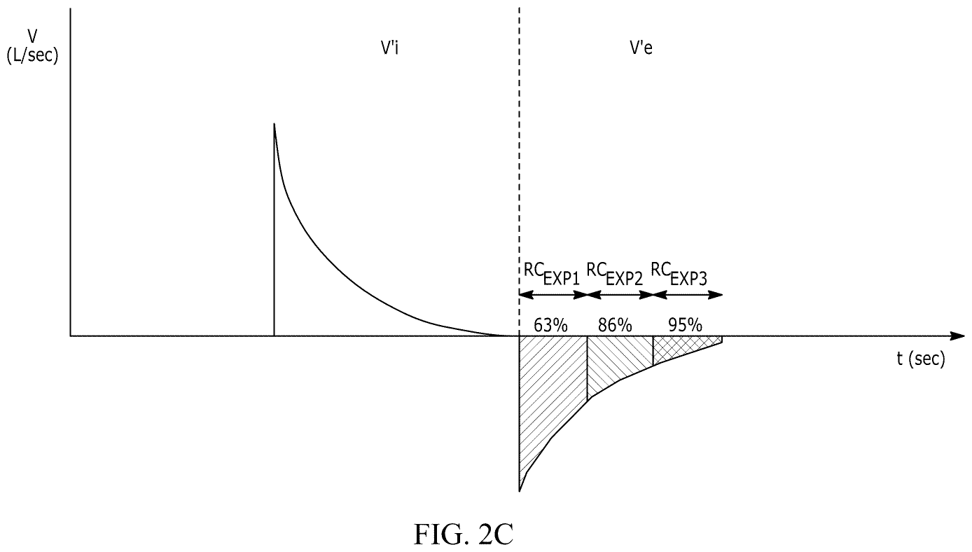

Time constant represents the time that a process takes to complete if its initial rate of change remains constant (e.g., linear process). Ventilation of the lung, however, is an exponential process and, by definition, time for lungs to empty after delivering Vt takes three time-constants (3×RC_{EXP}) (FIG. 2C). 3RC_{EXP} represents 95% of exhaled tidal volume and in clinical practice is considered a complete process. RC_{EXP} in respiratory mechanics is a product of compliance of the respiratory system (C_{RS}) and airway resistance (RAW). Inspiration and exhalation both operate with time constants, but expiratory RC_{EXP} is more representative as exhalation is presumed to be a passive process in mechanically ventilated patients. Additionally, measured RC_{EXP} is more representative than calculated values (RC_{EXP}=R*C) due to additional resistance caused by artificial airways, breathing circuits, humidification devices and the mechanical ventilator.

The first expiratory time constant represents the time by which 63% of end-inspiratory lung volume deflates (FIG. 2A, FIG. 2 B), the second time constant represents 86% of Vt exhaled, and the third 95% of Vt exhaled. This theory is generally recognized in healthy, homogenous lungs. However, in many cases the different time constants (for example, first, second, and third) are different due to physiologic and mechanical reasons. These reasons include but are not limited to premature airway closure and changes in resistance with varying flow. Therefore, there is a need to measure three RC_{EXP} from passive exhalation waveform and truly assess the exhaled volume at each TC (FIG. 2C). It is also useful to assess each of the time constants and determine a function of these time constants that may better represent the response and physiology of the patient and therefore allow for better selection of ventilator settings. This function of the time constants could include an average, median, minimum, maximum or other combination of the measured or estimated time constants. In this specification, it should be understood that the use of the word time constant can be any of the time constants or a function of the time constants described in this paragraph.

It should be noted that by definition, a system with a time constant increases or decreases by roughly 63% after the first time constant. It is standard clinical practice to estimate the time constant in a breathing patient. However, it is important to understand that similar results can be obtained by measuring parameters that are not, by definition, the “time constant”, but calculated similarly. For example, replacing the time constant with “time to exhale 50%, 60% or 70% of tidal volume” may have a similar value. Similarly, PEEP versus time constant plots can be evaluated with the second or third time constant as well. Lastly, a comparison of the 3 time constants or similar changes in R*C during the length of the exhalation can provide important information about the patient, including homogeneity of the lungs, presence of premature airway closure, presence of small or large airway disease, etc.

Time constant conclusions and advantages
1. There is a need to measure RC_{EXP}, not calculate it simply by multiplying R*C
2. All three time-constants-first, second and third may provide useful information, especially when compared in relation to their tidal volumes.
3. RC_{EXP} during PEEP titration may be more precise to evaluate exhalation dynamics due to respecting pulmonary mechanics in time (e.g. respecting small airways diameter change during exhalation) likely including also expiratory flow limitation (EFL).
4. Positive pressure in the airways during early exhalation may also be responsible for Vt redistribution, which cannot be assessed using static parameters during inspiratory (i.e. C_{RS}). Therefore, using measured RC_{EXP} may provide additional precision when assessing recruitment.

### Rationale of Using Alternating Levels of PEEP

Reference: Depta, F., Zdravkovic, M. & Gentile, M. A. Should we continue searching for the single best PEEP?. ICMx 10, 9 (2022), which is hereby incorporated by reference in its entirety.

Positive end-expiratory pressure (PEEP) has been an important component of mechanical ventilation for over 50 years, yet PEEP selection remains highly debatable in the critical care community. Despite numerous studies trying to answer the ‘PEEP question’, there are often conflicting opinions when it comes to recommendations based on clinical trials.

Three major studies (ALVEOLI, LOVS, EXPRESS) assessing higher versus lower PEEP in combination with low tidal volume (Vt) for acute respiratory distress syndrome (ARDS) failed to show a decrease in mortality, although a meta-analysis of the three trials suggested a survival benefit for a subgroup of patients with severe ARDS. It is universally accepted that higher PEEP may be helpful in ARDS patients where recruitability is expected but proves harmful in other groups of patients contributing to ventilation-induced lung injury (VILI). Therefore, as many authors have suggested that not only one PEEP doesn't fit all, but also looking for the ‘best’ PEEP has little meaning because, within mechanical power, it is just one part of the whole.

Since the introduction of PEEP into clinical practice, researchers have been focusing on finding the single most suitable PEEP that usually corresponds to the best lung mechanics (i.e., highest compliance of the respiratory system) or oxygenation. However, we would like to challenge this idea and introduce another approach to PEEP research in critically ill lungs. We hypothesize that patients with nonhomogeneous lungs could be supported on mechanical ventilation using multiple and alternating levels of PEEP. In the following paragraphs, we will list and elaborate on several reasons supporting our hypothesis of the potential benefits of using multiple alternating PEEP levels.

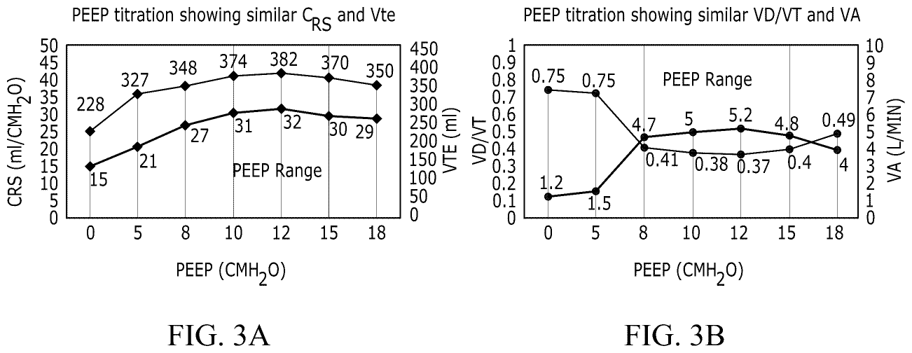

First, Suter demonstrated similar respiratory system compliance (C_{RS}) at different PEEP levels that depended on the Vt delivered. Data from our research on COVID-19-related ARDS identified rather a range of suitable PEEP levels where most parameters of lung mechanics during pressure-controlled ventilation show very similar values (FIG. 3). This range of PEEP levels was relatively wide: 5 to 8 cmH₂O. The range of suitable PEEP levels was also reported by other authors who assessed driving pressure using volume-controlled ventilation with different Vt (4, 6, and 8 ml/kg of predicted body weight [PBW]).

Second, the degree of recruitability varies widely in patients with ARDS. If constant inspiratory pressure is applied during the ascending PEEP levels and an increase in Vt is observed, lungs are considered recruitable. Given the previous studies and current knowledge, what PEEP level should be chosen if the lungs show evidence of similar recruitability at more than just one PEEP level? Furthermore, what deviation from the ‘best’ value is considered significant to guide the most appropriate PEEP selection? This is the range of suitable PEEP levels that show very similar values in the supposedly recruitable zone. Should the highest PEEP where best compliance is observed be selected, or rather the lowest PEEP with still satisfactory oxygenation?

Third, ventilation with a single PEEP level within the recruitable zone of ARDS lungs means that some alveoli will not be recruited, and some will be overdistended with respect to the pathophysiology of the ‘baby lungs’, including different regional time constants and opening airway pressures.

Fourth, alveolar dynamics continues to represent a “black box” of mechanical ventilation. Intra-tidal collapse has been rightfully feared to contribute to VILI. However, it has been shown that intra-tidal collapse and decollapse was similar at PEEPs of 5 and 15 cmH₂O in ARDS patients and that PEEP as high as 15 cmH₂O did not prevent cyclic alveolar opening and closing. Alveolar collapse happening during tidal ventilation with presumed sufficiently high PEEP is likely because the single best PEEP only represents the best tradeoff between recruitment and overdistension.

As Gattinoni et al. stated, “The best PEEP does not exist. To pretend and claim that we may find a PEEP level that avoids intra-tidal recruitment-derecruitment, providing in the meantime the best compliance, best oxygenation, and lowest dead space, without causing hyperinflation and affecting hemodynamics, reflects a wishful dream that has nothing to do with the reality”.

Perhaps alternating multiple levels of PEEP should be the parallel focus of research where similar lung mechanics are identified. Reasons to use alternating multiple levels of PEEP during mechanical ventilation while delivering controlled or supported breaths at each PEEP level, are:
1. It was shown on a 5-compartmental model of a nonhomogenous lung that the part of Vt is redistributed from shorter to longer time constant compartments when using multiple levels of PEEP. In clinical practice, this may represent better aeration and decrease in ventilation inhomogeneities.
2. Higher PEEP is not sustained and therefore, negative consequences (i.e., higher mechanical power delivered to the lung tissue or negative impact on hemodynamics) might not as pronounced.
3. If the recruitable zone of the ARDS lungs is larger than the delivered protective Vt (i.e., 6 ml/kg/PBW), then using constantly alternating multiple PEEP levels might allow improved recruitment without significantly increasing driving pressure (i.e., protective Vt is delivered at different PEEP levels thus ventilating bigger part of the recruitable zone of ARDS lung).

Therefore, if we acknowledge that intra-tidal collapse cannot be avoided and also know that ‘highest’ compliance, ‘best’ oxygenation and ‘lowest’ Vd/Vt, all occur at different PEEP levels, should research focus continue to seek the single best PEEP or rather consider a different approach? Targeting the recruitable zone of the nonhomogenous lungs using multiple alternating PEEP levels throughout the acute phase of ARDS, might propose an alternative approach on which to focus further research efforts (FIG. 3A and FIG. 3B).

### Description And Characteristics of TAPV Strategy

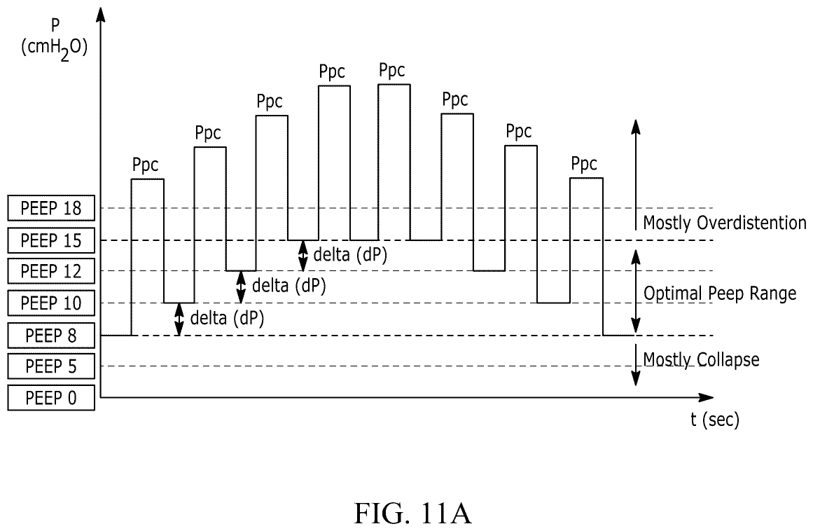

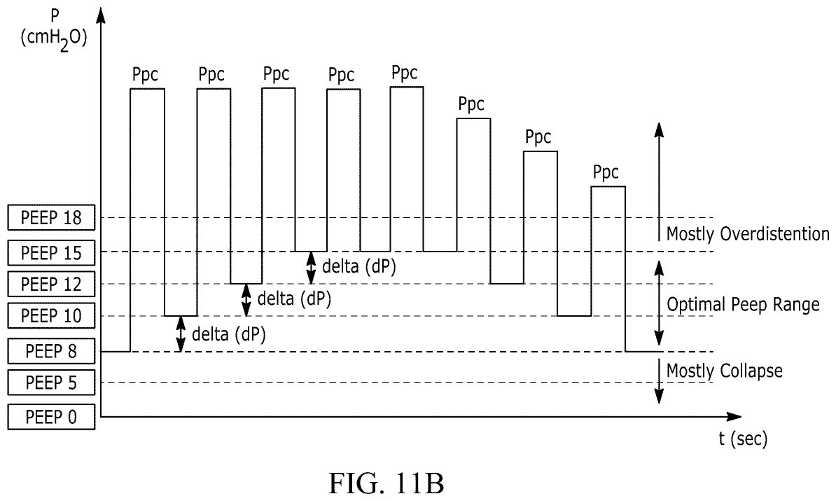

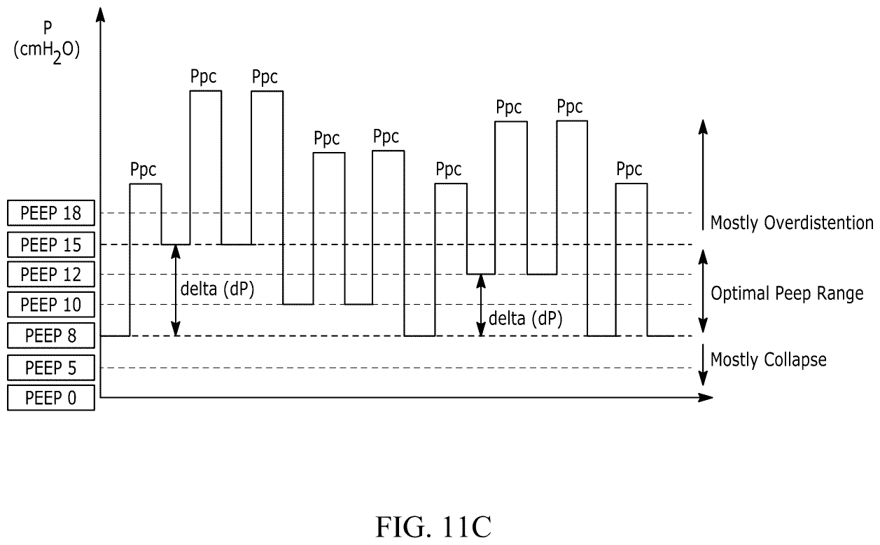

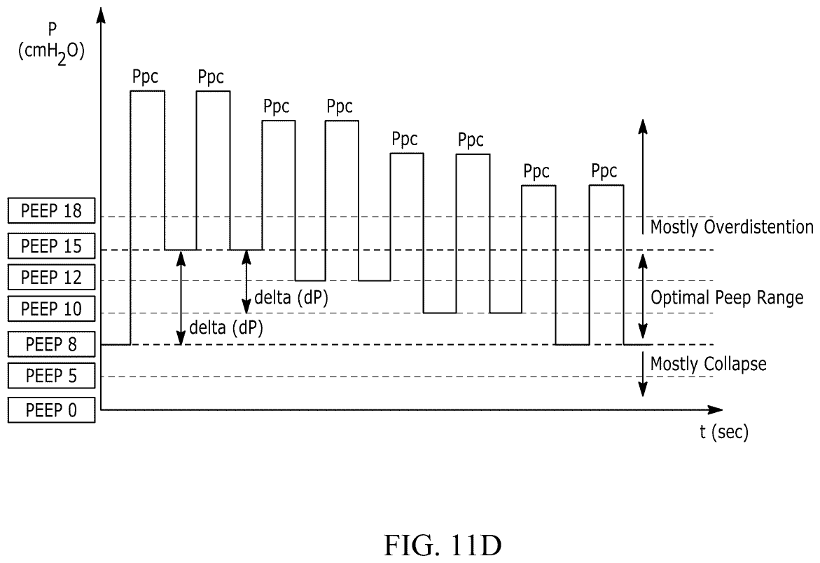

Basic characteristics of TAPV (Targeted alternating PEEP ventilation)

| | CHARACTERISTICS | RATIONALE |
|---|---|---|
| RC_{EXP} | Assessing a variety of PEEP levels to | Variable time will be spent on |
| diagnostics | assess changes in time constant at different | each PEEP until RCEXP stabilizes |
| (FIG. 4,5) | levels. This is sometimes called a PEEP | (±10 ms ), typically between 15 |
| | trial, but is not limited to strictly | to 60 seconds. Assessing the time |
| | incrementing or decrementing PEEP levels. | constant is faster than the time |
| | Typically, a wide range of PEEP levels are | required for a standard PEEP trial. |
| | evaluated, but only a local area can be used | Therefore the PEEP levels can be |
| | to determine if incremental changes in | changed rapidly to quickly |
| | PEEP can improve care. | determine the best settings. |
| Mode | Volume or pressure targeted modes (i.e. | All measurements are done using |
| | VCV, PCV, or similar). | VCV or PCV. |
| Inspiratory | For PCV - Set by the user with pressure | Default inspiratory pressure or |
| pressure or | limit (recommended starting pressure is 10- | tidal volume can be recommended |
| tidal volume | 14 cmH₂O), For VCV - protective Vt will | based on PBW or by measured |
| | be used 4-8 ml/kg/PBW | C_{RS} |
| Optimal | Use the time constant to determine required | Frequency can incrementally be |
| frequency | time to near complete exhalation to avoid | adjusted to minimize PEEPi as |
| (Fₚ) (FIG. 6) | air trapping/intrisnic PEEP (PEEPi). For | much as possible. |
| | example, using the first time constant, fₚ = | |
| | 60/(Ti + 3* RC_{EXP}). Other formulas based | |
| | on other time constants can be used based | |
| | on patient physiology. | |
| Max | Max multi-PEEP frequency = Fₚ + number | This is compensation for the time |
| frequency | of PEEP shifts (up and down) = total | lost during PEEP shifts |
| | optimal frequency adjusted to multi-level | |
| | setting | |
| PEEP | Evaluation of the RC_{EXP}, EI, or similar data | PEEP min − minimum PEEP of 4- |
| | allows for selection of the best PEEP or | 5 PEEP max − identified |
| | best PEEP range. Typically, the PEEP | according to the PEEP titration) |
| | levels that result in the maximum or | |
| | minimum (extreme) evaluation function are | |
| | selected as best or most acceptable PEEP | |
| | level. | |
| PEEP levels | 2 to 4 depending on RC_{EXP} pattern. | For NR patterns, predominantly 2 |
| | Number of PEEP levels may be selected | PEEP levels For R patterns −2 to |
| | based on the number of levels within a | 4 levels according to PEEP range. |
| | certain percentage of the best PEEP level. | PEEP levels can be adjusted by |
| | A graph of RC_{EXP}, EI or similar versus | increments of 2 cmH₂O manually |
| | PEEP elucidates how much these values | or set automatically using |
| | change with PEEP. In some cases nearby | automatic diagnostics |
| | PEEP levels are very similar and can all be | |
| | used. In other cases the curve is steeper and | |
| | using PEEP levels much different than the | |
| | best PEEP level may be detrimental. | |
| PEEP | Variable types possible depending on | Shifting type will be set based on |
| shifting | number of PEEP levels used | least mechanical power or driving |
| (FIG. 11A | | pressure (several concepts are |
| through FIG. | | possible), |
| 11D) | | |
| PEEP | RC_{EXP} or EI plots will be divided into | This aims to optimize minute |
| frequency | roughly equal clusters modified according | ventilation as much as possible. |
| | to the RC_{EXP} | Although mechanical properties |
| | | across PEEP range are similar, |
| | | they are not the same. Therefore, |
| | | PEEP shifting will be determined |
| | | based on the range of PEEP |
| | | levels. |
| FiO₂ | Amount of oxygen inspired by patient and | 100% FiO₂ is preferred for the |
| | adjusted by user. | whole measurement RC_{EXP} plot, |
| | | then adjusted by the operator to |
| | | optimize for patient need and |
| | | safety. |
| Trigger | Flow or pressure threshold to initiate a | Supported breath possible at any |
| | spontaneous breath | time during inspiration/ |
| | | exhalation |
| Rise time | set by the user in % or time | Should be adjusted to minimal |
| | | level to provide desired flow. |
| Cycling | Time or flow cycling for mandatory modes | Time or flow cycling to change |
| | | from inspiration to exhalation |
| Minute | Constant pressure or at each PEEP levels | Optionally adjusted by increasing |
| volume | will yield different Vt or PIP and therefore, | or decreasing inspiratory pressure |
| controller | controlling may be necessary to maintain | or Vtto match the set MV. |
| | stable MV | |

### The Best PEEP Determination, PEEP Range Levels and Mapping of the Recruitable Zone of the Nonhomogenous Lung

Healthy lungs are said to be homogenous, as different portions of the lungs have similar pulmonary mechanics. Therefore, these lungs are easy to ventilate and generally do not present a clinical problem. If PEEP titration is performed in patients and RC_{EXP} is evaluated or plotted against multiple PEEP levels, an RC_{EXP} plot (or more advanced Exhalation Index plot) can be obtained. Because healthy (homogenous) lung parenchyma has large functional residual capacity (compared to small to none FRC in ALI or ARDS), a plot showing RCexp as a parameter to assess lung recruitment will look very similar across most PEEP levels—i.e. RC_{EXP} plots will look almost flat (this is in contrast to RC_{EXP} (or exhalation index) plot in ALI or ARDS patients). Nonhomogeneous lungs will have different respiratory physiology in different regions of the lungs and therefore will have different RC_{EXP} values with different PEEP values. Therefore, the PEEP vs RC_{EXP} plot will be very different across PEEP levels.

Using pressure-controlled ventilation (PCV), we applied a different positive end-expiratory pressures (PEEP) and we measured RC_{EXP}, and other parameters (Vt, peak expiratory flow rate and deltaP) at each PEEP level (FIG. 5). Based on assessing these multiple variables, a multiple plots have been created for each patient and assessed in time (FIG. 4A through FIG. 4F). From that, an optimal PEEP and PEEP range can be estimated.

We used RC_{EXP} as a measured parameter during mechanical ventilation derived from expiratory flow curve (Ve) to determine PEEP and PEEP range levels (levels of PEEP where similar time constants are found±5% of maximum RC_{EXP} found) in mechanically ventilated patients with ARDS. Measurement of RC_{EXP} is expressed as time needed for exhalation of 63% of end-inspiratory tdal volume.

To create an evaluation parameter that reflects recruitment, speed of exhalation at given inspiratory pressure and overdistention at each PEEP level during PEEP titration we are proposing an “exhalation index” (EI) equation (1). EI is composed of all aspects of lung mechanics (pressure, volume and time) and represents a key component of ‘diagnostic’ PEEP titration into a complex formula:

$$EIs = Vt * {RC}_{EXPI} * Δ P \qquad ( 1 )$$

The first simplified EI may be referred to as the Simplified Exhalation Index (EIs) and the advanced formula below can be called Advanced EI (Ela). This can be upgraded and specified even more by adding a portion of tidal volume exhaled during first RC_{EXP} (i.e. 63% of exhaled Vt), and also adding peak expiratory flow rate (PEFR) to create even more precise formula (applying all four aspects of lung emptying—pressure, volume, flow and time), calculated at each PEEP level during PEEP titration:

$$EIa = ( V {t}_{63 \%} * {RC}_{EXPI} * Δ P ) / PEFR \qquad ( 2 )$$

Where Vt_{63%} is tidal volume exhaled during first RC_{EXP}, RC_{EXP1} is the first expiratory time constant, ΔP is the difference between Peak Inspiratory Pressure (PIP) and PEEP set on the ventilator and PEFR is peak expiratory flow rate at early at the beginning of exhalation. Using EI formulas, the range of PEEP levels will be considered as ±5-15% from maximum values (EI).

In nonhomogenous lungs, two patterns can be observed-recruitable (where increase in inspiratory pressure also causes increase in tidal volume) and nonrecruitable patterns (where the increase in pressure (or PEEP+Paw) does not cause an increase in tidal volume delivered.

Recruitable patterns mean that using constant inspiratory pressure, RC_{EXP} increases with increasing PEEP up to the maximum value (PEEPmax)| where best balance between recruitment and overdistention is identified. Then, RC_{EXP} decreases, which suggests hyperinflation of the lungs and is generally unwanted.

A nonrecruitable pattern is usually present in very severe forms of Acute Lung Injury (ALI) and Acute Respiratory Distress Syndrome (ARDS). This is a form of lung affection where only small portion of lungs is responsible for gas exchange. On RC_{EXP} plot (or using EI plot) RC_{EXP} shows highest values at low PEEP levels and progressively decreases with increasing PEEP levels. The reason for that is overdistention of small portion of remaining recruitable lung tissue that is being overdistended and “pushes back” faster.

Therefore, RC_{EXP} can be used as an indicator for lung recruitability using PEEP titration metod, either as a sole parameter or, better, in combination with other parameters of pulmonary exhalation mechanics.

### We Performed a Clinical Trial:

Background: We hypothesized that the measured expiratory time constant (RC_{EXP}) could be a bedside parameter for the evaluation of positive end-expiratory pressure (PEEP) settings in mechanically ventilated COVID-19 patients during pressure-controlled ventilation (PCV).

Methods: A prospective study was conducted including consecutively admitted adults (n=16) with COVID-19-related ARDS requiring mechanical ventilation. Ventilator settings for all patients included PCV, 18 breaths per minute, inspiratory pressure 14 cmH₂O, I:E ratio 1:1.5 and FiO₂ 1.0. RC_{EXP} was measured and recorded at each PEEP level (0 to 18 cmH₂O) in prone (n=29) or supine (n=24) positions. RC_{EXP} plots were created to graphically show changes in individual physiology with PEEP. The PEEP setting with the highest RC_{EXP} (RC_{EXP} (MAX)) was considered to represent the best tradeoff between recruitment and overdistention. The PEEP range was calculated as ±5% from the RC_{EXP} (MAX).

Results: Two groups of patterns were observed in the RC_{EXP} plots: recruitable (R) (75%) and non-recruitable (NR) (25%). In the R group, the optimal PEEP and PEEP ranges were 8±3 cmH₂O and 6-10 cmH₂O for the prone position and 9±3 cmH₂O and 7-12 cmH₂O for the supine position. In the NR group, the optimal PEEP and PEEP ranges were 4±4 cmH₂O and 1-8 cmH₂O for the prone position and 5±3 cmH₂O and 1-7 cmH₂O for the supine position, respectively. The R group showed significantly higher optimal PEEP (p<0.004) and PEEP ranges (p<0.001) than the NR group. Forty-five percent of measurements resulted in the most optimal PEEP being significantly different between the positions (p<0.01).

Conclusions: Based on RC_{EXP} plots, responses to PEEP were easily measured. There was wide variation in patient responses to PEEP, which indicates the need for personalized evaluation.

Based on the results of these study, we measured RC_{EXP}, tidal volume (Vt), delta pressure (difference between PIP and PEEP during exhalation) and Peak Expiratory Flow Rate (PEFR) during exhalation at each PEEP level a created EI parameter—EI=(Vt_{63%}*RC_{EXP1}*ΔP)/PEFR.

### Measurement And Evaluation Procedure

a. The mechanical ventilator will prompt the clinician to ensure the patient is “passive” (i.e. without spontaneous breath activity and without triggering)
b. When pressed “TEST” the device automatically starts preoxygenation with 100% O₂ in the original mode for 2 minutes (which is standard for most ventilators, but can be varied). Oxygen will remain at 100% for the entire time to ensure safety. To ensure safety, this maneuver will be canceled by any patient respiratory effort.
c. After completion of preoxygenation it sets parameters for PEEP titration: PCV or VCV mode, positive end-expiration pressure (PEEP) 15-20 cmH₂O based on prior user selection, constant PCV between 10-18 cmH₂O or VCV at protective Vt of 4 to 8 ml/kg/PBW based on prior user selection, frequency 10-20/min depending on user selection before, inspiratory time 33% of breathing cycle (i.e., I:E ratio of 1:2) (FIG. 5)
d. It then gradually changes PEEP values in a range from 5 to 20 cmH₂O by increments of 2 to 3 cmH₂O based on prior user selection. At each PEEP level, 3-20 breath cycles are delivered. These settings are configurable by clinicians. Alternatively, the ventilator' programmed algorithm will deliver a variable number of breaths depending on lung mechanics found.
e. Records the value of the expiration time constant (RC_{EXP}) and makes the average of the values from the last 3-20 breath cycles at each PEEP level.
f. RC_{EXP}, PEFR, Vt and deltaP values are recorded.
g. Next, an equation to calculate an exhalation dynamics parameters (reflecting recruitment, speed of exhalation and overdistention) at each PEEP using EXHALATION INDEX: EIa=(Vt_{63%}*RC_{EXP1}*ΔP)/PEFR or simplified equation EIs=Vt*RC_{EXP1}*ΔP
h. From the Exhalation Index (Ela or EIs) plots, it determines recruitability-recruitable or nonrecruitable patterns.
i. The system calculates acceptable or optimal PEEP level(s) and PEEP range levels that will be displayed along with recommendation how many PEEP levels should be selected. The system may also automatically select or implement these PEEP levels. When multiple PEEP levels are in the acceptable range, the ventilator may automatically modify the PEEP through this range breath to breath or every few breaths to obtain the advantages provided by using multiple PEEP levels.
j. There are a small percentage of patients for which it is unlikely to be possible to automatically determine the ideal range of PEEP values. In this case, the PEEP levels will need to be chosen by the clinician.

### Brief Summary of the Invention and Possible Advantages of this Method

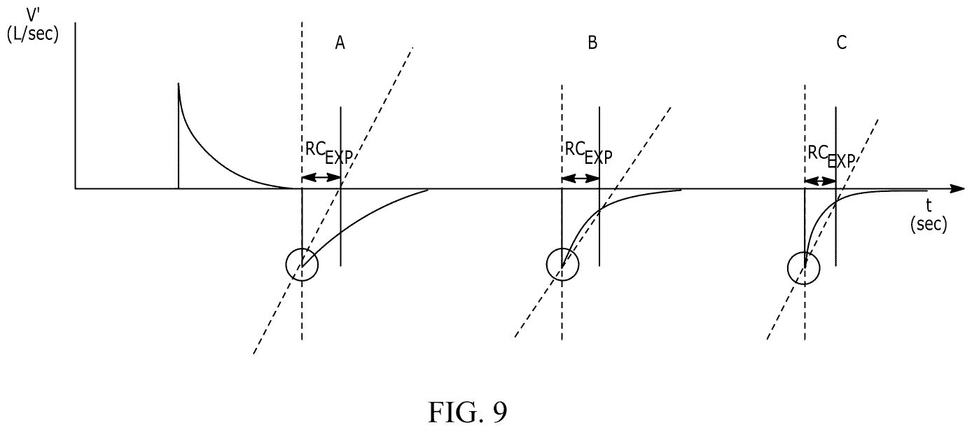

a. More precise optimization of PEEP and PEEP range for each patient based on measured variables (RC_{EXP}), functions of multiple time constants, and functions including time constants and other measured lung variables (i.e., proposed exhalation angle-FIG. 9).
b. Assessing lung homogeneity/inhomogeneity
c. The method is non-invasive, applied bedside, is repeatable, with no radiation exposure, no transport of the patient is required, and no other hardware is necessary (FIG. 5).

### Determine the Maximum Optimal Frequency, Optimal Cycle Time, Optimal I:E Ratio And Optimal Complete Exhalation Time

### BACKGROUND: Predicting the Optimal Ventilation Frequency and Adjusting it to Minimize Intrinsic PEEP

Respiratory rate is one of the key variables to set and monitor during mechanical ventilation. Its potentially harmful effects have been recently recognized, as excessive respiratory rate, per se, can cause ventilator-induced lung injury (VILI). This is mainly caused by incomplete exhalation, which is expressed on mechanical ventilators as intrinsic PEEP (PEEPi). One goal of the invention is to find the optimal frequency to balance/maximize minute ventilation while preventing PEEPi as much as possible. Similarly, optimal I:E ratio, total time of the respiratory cycle as well as complete exhalation time has been identified.

Based on clinical research, we compared the respiratory rate with measured RC_{EXP} in relation to PEEPi. We predicted the optimal predicted respiratory rate (f_{P}), optimal predicted time respiratory cycle time (T_{CYP}), and optimal predicted I:E ratio (IE_{P}), which has been defined as predicted respiratory rate, time cycle and I:E ratio where PEEPi is approaches zero. This is based under the assumption that three time-constants represent full exhalation and that inspiratory flow pattern remains constant.

Optimal predicted respiratory cycle time (T_{CYP}) incorporating time constant can be expressed using the following equation (Equation 1) (FIG. 6):

$${T}_{CYP} = Ti + 3 * {RC}_{EX P} \qquad ( 1 )$$

Where T_{CYP} is the predicted respiratory cycle time, Ti represents inspiratory time and RC_{EXP} represents the measured first expiratory time constant. When the time constants are not equal, other formulas based on functions of the time constants (for example, minimum, maximum or average) can also be used. Based on these methods, one can estimate the predicted respiratory rate (f_{P}) at which complete exhalation is anticipated (Equations 2 and 3):

$${f}_{P} = 60 / {T}_{CYP} \qquad ( 2 )$$

$${f}_{P} = 60 / ( Ti + 3 * {RC}_{EX P} ) \qquad ( 3 )$$

where f_{P} is a predicted respiratory rate when PEEPi is close to 0 cmH₂O, Ti is inspiratory time(s) and RC_{EXP} is first expiratory time constant(s) measured from expiratory flow waveform.

Similarly, I:E ratio can be calculated based on measured RC_{EXP} to determine optimal I:E ratio where PEEPi approaches 0 cmH₂O using the following equation (Equation 4):

$${IE}_{P} = Ti / 3 * {RC}_{EX P} \qquad ( 4 )$$

Where IE_{P} is predicted I:E ratio, Ti represents inspiratory time and RC_{EXP} is expiratory time constant.

I:E ratio can also be optimized in cases where the optimal respiratory rate has been determined but is inapplicable in real clinical scenarios (i.e. higher frequencies are used). Then, I:E ratio may be altered using RC_{EXP} to allow shorter inspiration and prolonged exhalation in order to avoid high PEEPi.

Predicted respiratory rate (f_{P}), T_{CYP} and IE_{P} was calculated for a subgroup of passive patients ventilated with mandatory PCV with the same Ti set to 40% (I:E of 1:1.5) where PEEPi was more than 2 cmH₂O. Measured and predicted respiratory rates were also compared and assessed statistically (mean SD for those where RC_{EXP} was both short and long, p values).

Similarly, formula to determine complete exhalation (T_{CE}) without using inspiratory time (Ti) can be used by correlating exhalation time (Te) with 3×RC_{EXP} as in the following formula:

$${T}_{CE} = Te / 3 * {RC}_{EX P} \qquad ( 5 )$$

Where t_{CE} is complete exhalation time, Te is exhalation time, RC_{EXP} is the first expiratory time constant(s) measured from expiratory flow waveform, and f is the respiratory rate

| | |
|---|---|
| a. | If Te/3* RC_{EXP} > 1 then the respiratory rate may be increased to |
| | maximize the required minute volume (MV) |
| b. | If Te/3* RC_{EXP} = 1, then exhalation time is optimally set |
| c. | If Te/3* RC_{EXP} < 1 then the total respiratory rate may be decreased |
| | to prevent negative consequences of PEEPi. |

In case the PEEPi remains unacceptably high despite using optimization as in (1) and (2), then change in inspiration-to-expiration (I:E) ratio will be required to decrease inspiratory time (Ti) and prolong exhalation. Both equations (1) and (2) will be used to optimize I:E ratio in a way so the PEEPi can return to acceptable levels (FIG. 6).

### Verify Recruitability By Performing Series of Inspiratory Holds of Different Duration

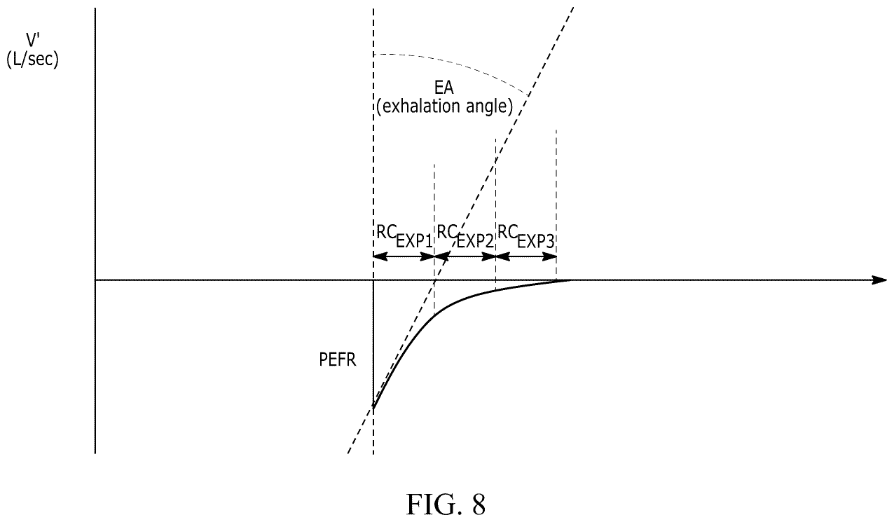

Background: Identifying recruitability provides a “safe region” to ventilate nonhomogenous lungs. Therefore, its identification by performing PEEP titration will provide the distending pulmonary pressure margins. If non-recruitability is identified, it can be verified by the series of inspiratory holds of different duration to prove that nonhomogenous lungs cannot be recruited even if longer inspiration is provided (FIG. 8 and FIG. 9). RC_{EXP} might also be used to confirm the severity of recruitability/non-recruitability in mechanically ventilated patients.

### Brief Summary of The Invention And Possible Advantages of This Method

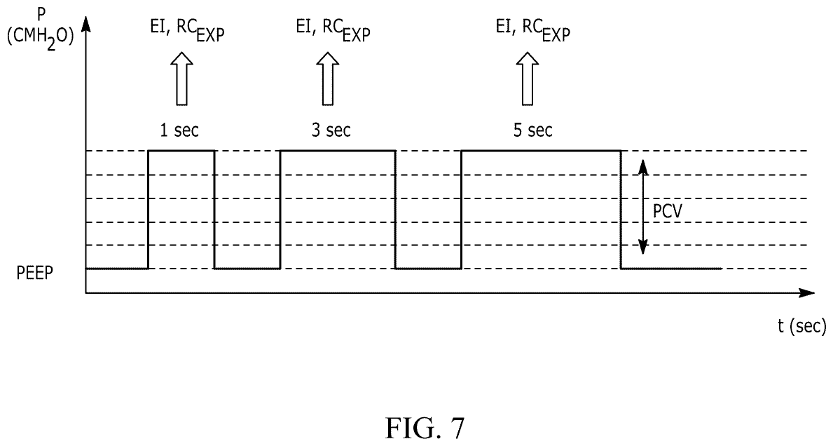

The ventilator will deliver a series of at least 3 mechanical breaths at clinician selected PEEP level (ideally the best PEEP), where each has different or longer inspiratory times or extended end-inspiratory pauses (i.e., inspiratory hold for 1, 2, and 4 seconds or 2, 4 and 6 seconds etc.) and the system compares the RC_{EXP} at the longer and shorter inspiratory times or end-inspiratory pauses to identify whether RC_{EXP} is significantly different in each of these breaths. If RC_{EXP} changes significantly, then there is still assumed recruitability. If RC_{EXP} does not change significantly, then even long inspiration will not redistribute gas into more diseased areas of the lung (longer time constants), and the lungs are considered non-recruitable. By confirming recruitability, the ventilator will further adjust the PEEP settings (FIG. 7).

### Calculating Exhalation Angle And Expiratory Flow-To-Time Constant Ratio (PEFRr: RC_{EXP} Ratio) To Determine Inappropriately High PEEP Levels

BACKGROUND: When higher PEEP levels are delivered, overdistension is a very probable for ALI and ARDS patients, most notably for those who show very little recruitability. Therefore, knowing peak expiratory flow rate at the beginning of exhalation and expiratory flow at first, second and, third time constants may provide useful information about exhalation dynamics. More, it may show how fast the flow decreasing between EFR at RC_{EXP1}, EFR at RC_{EXP2} and EFR and RC_{EXP3}.

### Brief Summary of The Invention And Possible Advantages of This Method

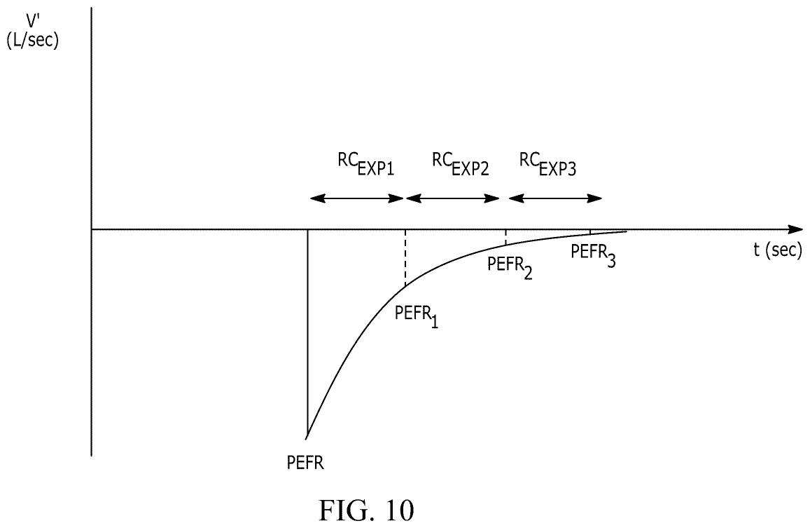

Exhalation angle (EA) and PEFR1, PEFR2 and PEFR3 may provide useful information when inappropriately high PEEP is selected or when airways obstruction is present (FIG. 8, FIG. 9 and FIG. 10).

### Determine the Most Appropriate PEEP Shifting After Ideal PEEP Range Has Been Identified

BACKGROUND: When alternating PEEP levels are employed during mandatory or supported ventilation, most optimal PEEP shift will be determined. There may be 2, 3 or even 4 different and constantly alternating PEEP levels depending on the width of PEEP range identified based on ‘diagnostic’ PEEP titration suggesting a recruitable zone of the lung (FIG. 11-FIG. 14).

### Brief Summary of The Invention And Possible Advantages of This Method

Numbers of PEEP levels that were identified should alternate based on:
1. minimum energy expenditure (e.g., the lowest possible mechanical energy and mechanical power during PEEP shifting).
2. We also propose to test different PEEP shifting strategies to maximize Vt redistribution while selecting the most efficient pattern. This will be done using a physiological computer model that we are currently developing. (FIG. 11)

The ventilator will automatically select and evaluate various PEEP titrations in an additional embodiment by assessing the physiologic response to the settings. The physiologic response may include assessment of improvements to CO2 elimination, oxygen uptake, lung and airway assessments such as dead space, resistance and compliance, mechanical power/energy, and other methods. This data would be assessed to determine the best possible PEEP titration. In many situations, an optimal solution will be difficult to assess because different parameters may be optimized by different settings. In such cases, a complex or intelligent algorithm may be trained and utilized to determine the best settings based on patient characteristics and patient responses to ventilation.

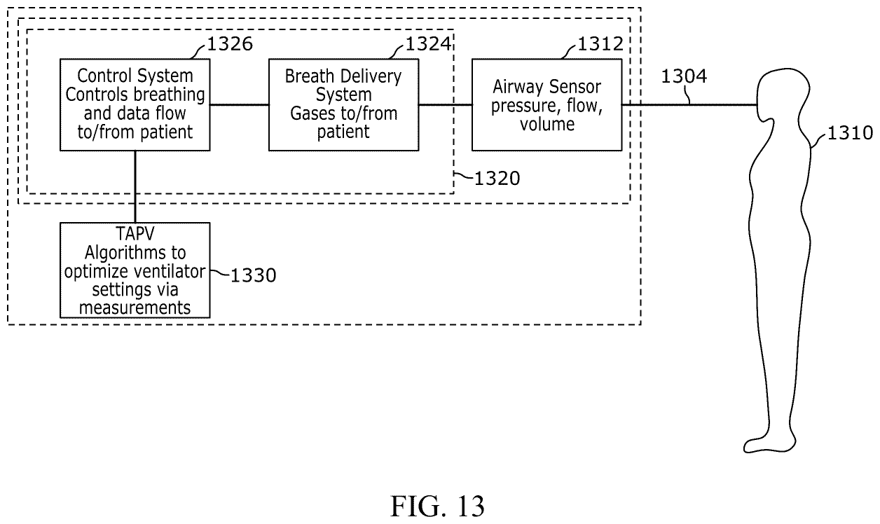

FIG. 13 is a diagram of the system illustrating the sensors, which are connected either wirelessly or wired to a module that transmits the data to a computer or ventilator. The system includes airway sensors 1312, such as but not limited to, an esophageal pressure sensor, airway pressure and flow sensor, and other optional sensors, including CO2 sensor, pulse-oximetry sensor, and blood pressure sensor on a patient. The sensors 1312 are connected either wirelessly or wired to a computer, mobile device, web-based device or service, or other computing system or TAPV box or ventilator 1320 that can implement the computer decision support and algorithms described herein. The computing device accepts the data from the sensor transmission module along with data from the clinician via a user interface (e.g., patient position) and possibly data from the electronic medical record (patient diagnosis, patient medications, patient lung health, patient demographics such as height, etc.), physiologic monitor (blood pressure, Co2, ECG, etc.), and/or ventilator (ventilator sensors or settings). A control system 1326, and a breath delivery system 1324, work cooperatively with the algorithm 1330. The algorithm 1330 code can run on a mobile phone, tablet, laptop, or desktop computer or as a web service when connected to the sensors and a method of receiving data from those sensors. The algorithms, sensors, control system 1326, and breath delivery system 1324 can all be built into a single medical device that functions as a smart respiratory monitor or as part of a ventilator. In another example, ventilator 1320 is capable of delivering gases to and from the patient, 1330 is a computer, mobile device, computing system, etc., capable of implementing these algorithms and providing advice to the clinician or directly controlling the ventilator, and 1340 (the outside box) is an integrated system that can implement all these functions.

### NON-LIMITING EXAMPLES

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the disclosure, skilled artisans will appreciate that certain features are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A system for mechanically ventilating a patient comprising:
a breath delivery system;
at least one airway sensor; and
a computing means, wherein the breath delivery system provides a flow of gas to the patient, the at least one airway sensor measures pressure and flow of the gas leaving the patient, the computing means calculates an expiratory time constant, and the computing means evaluates lung recruitability of the patient using the expiratory time constant (RC_{EXP})
wherein a PEEP level provided to the patient is automatically modified by the system to span a range of PEEP levels.

2. The system of claim 1, wherein the breath delivery system provides one or more breaths with longer inspiratory times while maintaining tidal volume or constant pressure delivered to the patient and the computing means compares the expiratory time constant with a longer inspiratory time to the expiratory time constant with shorter inspiratory time at a same inspiratory pressure.

3. The system of claim 2, wherein the longer inspiratory time is obtained by adding an end inspiratory pause.

4. The system of claim 2 or 3, wherein the lung recruitability is higher when the expiratory time constant is higher with longer inspiratory time.

5. (canceled)

6. (canceled)

7. (canceled)

8. The system of claim 1, wherein PEEP levels are determined based on larger values of variables assessing recruitability.

9. The system of claim 8, wherein a range of PEEP levels is determined by the PEEP levels that have highest values of variables assessing recruitability within a certain percentage of a maximum expiratory time constant.

10. The system of claim 1, wherein one or more PEEP settings are maintained until a measurement of a variable assessing recruitability is stable, which is preferably no more than 20 breaths, and most preferably no more than 5 breaths.

11. The system of claim 10, wherein the system provides an indication of when the measurement of expiratory time constant is stable so that the PEEP settings can be changed.

12. The system of any one of claims 10-11, wherein the system automatically changes PEEP settings when a measurement of any variables assessing optimal PEEP levels is stable.

13. A system for mechanically ventilating a patient comprising:
a breath delivery system;
at least one airway sensor; and
a computing means, wherein the breath delivery system provides a flow of gas to the patient, the at least one airway sensor measures pressure and flow of the gas leaving the patient, the computing means calculates an expiratory time constant, and the computing means evaluates lung homogeneity of the patient using the expiratory time constant;
wherein the breath delivery system provides PEEP to the patient and wherein the computing means determines homogeneity of the lungs by evaluating a change in multiple variables or functions of the expiratory time constant at a range of PEEP levels.

14. (canceled)

15. The system of claim 13, wherein the range of PEEP levels provided to a patient is automatically modified by the system to span several PEEP levels in the range.

16. The system of claim 13, wherein a maximum expiratory time constant is computed across a range of PEEP levels and lung nonhomogeneity is determined when expiratory time constants across a range of PEEP levels vary by more than a certain percentage of the maximum expiratory time constant, preferably within one of 5%, 10%, 15%, or 20%.

17. (canceled)

18. The system of claim 1, wherein a range of PEEP levels provided to the patient is automatically modified by the system to span several PEEP levels in a preselected range.

19. The system of claim 1, wherein the computing means calculates one or more values of an exhaled tidal volume, expiratory flow rate and driving pressure, and combines the expiratory time constant with one or more of the values to create the index of acceptability.

20. The system of any one of claims 18-19, wherein the computing means creates an index to evaluate an acceptability of a range of one or more PEEP levels based on the expiratory time constant or functions of the expiratory time constant and wherein the index to evaluate an acceptability of a PEEP level is based on a function of multiple variables and the function is multiplication or addition.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The system of claim 1, wherein the lung recruitability is determined by a constant inspiratory pressure being applied during a decremental PEEP levels and an increase in Vt is observed.
